(19) United States
(12) Patent Application Publication
(10) Pub. No.: US 2020/0148762 A1
(43) Pub. Date: May 14, 2020

(54) METHODS FOR REGULATING ENDOGENOUS PRODUCTION OF ANTIBODIES AGAINST INFECTIOUS DISEASES

(71) Applicant: Avamab Pharma Inc., Calgary (CA)

(72) Inventors: Laura Van Lieshout, Calgary (CA); Sarah Wootton, Calgary (CA); Bradley G. Thompson, Calgary (CA)

(21) Appl. No.: 16/185,598

(22) Filed: Nov. 9, 2018

(51) Int. Cl. C07K 16/28; C12N 15/85; C12N 15/62; A61P 31/04; A61P 31/10; A61P 31/12; A61P 33/02; C07K 16/20; C07K 16/12; A61K 47/68

(52) U.S. Cl. CPC C07K 16/28; C12N 15/85; C12N 15/62; A61P 31/04; A61P 31/10; A61P 31/12; A61K 47/6879; C07K 16/20; C07K 16/12; A61K 47/6839; A61K 47/6843; A61K 47/6849; A61P 33/02

(57) ABSTRACT

The present disclosure relates to the composition of one or more agents, therapies, treatments, and methods of use of the agents and/or therapies and/or treatments for upregulating production of two or more antibodies, one or more bi-specific antibodies, or combinations thereof. Embodiments of the present disclosure can be used as a therapy or a treatment of a conditions including: sepsis, parasites, and active and chronic infections caused by bacteria, non-hemorrhagic viruses, amoeba, mycoplasma, fungus, prions or combinations thereof.

### TECHNICAL FIELD

The present disclosure generally relates to viral gene vectors engineered to endogenously produce antibodies against infectious diseases. In particular, the present disclosure relates to agents, therapies, and methods of use of the agents and/or therapies for upregulating endogenous production of two or more of antibodies, one or more bispecific antibodies, and combinations thereof.

### BACKGROUND

Antibodies are proteins that bind to a variety of molecules recognized as foreign by the immune system.

Recombinant antibodies are known to be used to treat infectious disease, cancers, autoimmune diseases and are used as antisera against bacterial toxins.

Endogenously produced antibodies induced by vaccinations protect against infectious disease agents. Viral gene vectors that endogenously produced single antibodies have been suggested as treatments for active infectious disease agents. However, two or more endogenously produced antibodies, one or more endogenously produced bispecific antibodies, and combinations thereof have not been used to treat certain active infectious diseases.

### SUMMARY

Embodiments of the present disclosure relate to a method for inducing endogenous production of antibodies against infectious diseases by using one or more gene vectors that contain nucleotide sequences and/or genes that encode for the production of two or more antibodies, one or more bispecific antibodies, or combinations thereof.

Some embodiments of the present disclosure relate to a method of making an agent/target cell complex. The method comprises a step of administering a therapeutically effective amount of the agent to a subject, wherein the agent/target cell complex may increase the subject's production of two or more antibodies, one or more bispecific antibodies, or combinations thereof.

Some embodiments of the present disclosure relate to a method of making an agent/target cell complex, the method comprising a step of administering a sufficient amount of an agent to a target cell whereby the agent/target cell complex is formed, wherein the agent/target cell complex may increase the production of two or more antibodies, one or more bispecific antibodies, or combinations thereof.

Some embodiments of the present disclosure relate to a pharmaceutical agent that comprises an agent, a pharmaceutically acceptable carrier, and/or an excipient. The agent may cause upregulate the production of two or more antibodies, one or more bispecific antibodies, or combinations thereof.

Some embodiments of the present disclosure relate to a kit used for treatment of a condition or for delivery of a therapy to a subject. The kit comprises a unit dosage of an agent, a carrier for the unit dosage, and instructions for administering the unit dosage to the subject. The agent may upregulate production of two or more antibodies, one or more bispecific antibodies, or combinations thereof. The carrier may be a solid carrier, such as a capsule or tablet, or a liquid carrier or other fluid carrier. The instructions may describe how the carrier may be administered to a subject for an optimal effect. The instructions may also describe how the carrier may be administered to a subject by various routes of administration.

Some embodiments of the present disclosure relate to a method of treating a condition. The method comprises a step of administering to a subject a therapeutically effective amount of an agent that upregulates a production of two or more antibodies, one or more bispecific antibodies, or combinations thereof. Without being bound by any particular theory, embodiments of the present disclosure may be useful for treating conditions including sepsis, parasites, active and chronic infections caused by bacteria, non-hemorrhagic viruses, ameoba, mycoplasma, fungus, and prions, and preventing active and chronic infections in immunocompromised patients, and in patients with immature immune systems.

### DETAILED DESCRIPTION

### Definitions

Unless defined otherwise, all technical and scientific terms used herein have the meanings that would be commonly understood by one of skill in the art in the context of the present specification. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, of a given value or range. It is understood that such a variation is always included in any given value provided herein, whether or not it is specifically referred to.

As used herein, the term “agent” refers to a substance that, when administered to a patient, causes one or more chemical reactions and/or one or more physical reactions and/or or one or more physiologic reactions in the patient.

As used herein, the term “cell” refers to a single cell as well as a plurality of cells or a population of the same cell type or different cell types. Administering an agent to a cell includes in vivo, in vitro and ex vivo administrations or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, or any post-translational modifications of one or more proteins.

As used herein, the term “excipient” refers to any substance, not itself an agent, which may be used as a component within a pharmaceutical composition or a medicament for administration of a therapeutically effective amount of the agent to a subject. Additionally or alternatively, an excipient may alone, or in combination with further chemical components, improve the handling and/or storage properties and/or to permit or facilitate formation of a dose unit of the agent. Excipients include, but are not limited to, one or more of: a binder, a disintegrant, a diluent, a buffer, a solvent, a thickening agent, a gelling agent, a penetration enhancer, a solubilizing agent, a wetting agent, an antioxidant, a preservative, a surface active agent, a lubricant, an emollient, a substance added to improve the appearance or texture of the composition, and a substance used to form the pharmaceutical compositions or medicaments. Any such excipients can be used in any dosage forms according to the present disclosure. The foregoing classes of excipients are not meant to be exhaustive but are provided merely as illustrative of what a person of skill in the art would know; a person of skill in the art would also recognize that additional types and combinations of excipients may be used to achieve delivery of a therapeutically effective amount of the agent to a subject through one or more routes of administration.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition for administration of the agent to a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render the formulation isotonic. Examples of suitable pharmaceutically acceptable carriers include, but are not limited to: saline solutions, glycerol solutions, ethanol, N-(1(2, 3 -dioleyloxy)propyl)-N,N,N-trimethylammonium chloride (DOTMA), diolesylphosphotidylethanolamine (DOPE), and liposomes of various constituents. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require that the formulation incorporate enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding an onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of amino acid sequences, and/or the production or functionality of one or more regulatory molecules that can influence the production or functionality of an effector molecule.

As used herein, the terms “promote”, “promotion”, and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence of and/or the progression of the disease or disorder within the subject.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism; an in vitro culture of one or more tissue types, an in vitro culture of one or more cell types; ex vivo preparations; and a sample of biological materials such as tissue and/or biological fluids.

As used herein, the term “target cell” refers to one or more cells that are deleteriously affected, either directly or indirectly, by an infection.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or may be therapeutic in providing a partial or complete amelioration or inhibition of a disease, disorder, or symptom thereof. Additionally, the term “treatment” refers to any treatment of a disease, disorder, or symptom thereof in a subject and includes: (a) inhibiting the disease, i.e., arresting its development; and (b) ameliorating the disease.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent, and the severity of the disease, disorder or symptom thereof. The subject's age, weight and genetic make-up may also influence the amount of the agent that will be a therapeutically effective amount.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active-ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 2%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments and methods of use of the agents and/or therapies and/or treatments for upregulating production of two or more antibodies, one or more bispecific antibodies, and combinations thereof. Some embodiments of the present disclosure relate to methods for making a complex between at least one particle of an agent and at least one target cell of a subject for upregulating that subject's production of two or more antibodies, one or more bispecific antibodies, and combinations thereof.

In some embodiments of the present disclosure, the agent can be administered to the subject by an intravenous route, an intramuscular route, an intraocular route, an intraperitoneal route, an intrathecal route, an intravesical route, a topical route, an intranasal route, a transmucosal route, a pulmonary route, an oral route and combinations thereof.

In some embodiments of the present disclosure, the agent can be administered to the subject by pipetting a dose of the agent into an in vitro cell culture, perfusing or immersing an ex vivo cell or tissue preparation with a solution that comprises the agent, mixing a biological fluid sample with a solution or substrate that comprises the agent, or combinations thereof.

Some embodiments of the present disclosure relate to an agent that can be administered to a subject with a condition that includes but is not limited to: sepsis, parasites, and active and chronic infections caused by bacteria, viruses, amoeba, mycoplasma, fungus, and prions. When a therapeutically effective amount of the agent is administered to the subject, the subject may change production and/or functionality of one or more immune-system molecules. For example, the subject may increase production of an antibody by changing the production of one or more sequences of DNA, one or more sequences of RNA and/or one or more proteins and/or one or more regulatory molecules that regulate the subject's levels and/or functionality of two or more antibodies, one or more bispecific antibodies, and combinations thereof.

In some embodiments of the present disclosure, the subject may respond to receiving the therapeutic amount of the agent by changing production and/or functionality of two or more antibodies, one or more bispecific antibodies, and combinations thereof by changing production and/or functionality of one or more DNA sequences, one or more RNA sequences, and/or one or more proteins that regulate the levels and/or functionality of one or more intermediary molecules. The one or more intermediary molecules regulate the subject's levels and/or functionality of two or more antibodies, one or more bispecific antibodies, and combinations thereof.

In some embodiments of the present disclosure, the agent can be: a vector used for gene therapy; one or more selected nucleotides, a sequence of nucleotides, one or more nucleosides, a sequence of nucleosides, a RNA complex, a DNA complex or combinations thereof.

In some embodiments of the present disclosure, the agent is a vector that comprises a gene insert, for example a recombinant virus vector (RVV), used for gene therapy. The gene therapy is useful for increasing the production of two or more antibodies, one or more bispecific antibodies, and combinations thereof. For example, the RVV can induce a target cell to increase production of two or more antibodies, one or more bispecific antibodies, and combinations thereof.

In some embodiments of the present disclosure, the agent is a virus that can be within one or more of the following genus: flavivirus, influenza, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, rubulavirus, simplexvirus, varicellovirus, vesiculovirus, lyssavirus, cytomegalovirus, or combinations thereof.

Some embodiments of the present disclosure also relate to administering a therapeutically effective amount of the agent. The therapeutically effective amount of the agent will not substantially increase or advance any deleterious conditions within the subject. For example, the therapeutically effective amount will not cause cytokinesis, hypercytokinemia, or any other uncontrolled, or partially controlled, upregulation of the subject's immune system. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments the therapeutically effective amount of the agent is between about 10 and about 1×10¹⁶ TCP/kg. In some embodiments of the present disclosure, the therapeutically effective amount of the agent that is administered to a patient is measured in VG/kg (total viral genome of the agent per kilogram of the patient's body weight). In some embodiments the therapeutically effective amount of the agent is between about 10 and about 1×10¹⁶ VG/kg.

Some embodiments of the present disclosure relate to a method for making a complex within a subject. The method comprises a step of administering a therapeutically effective amount of the agent to the subject. The complex comprises at least one particle of the agent, and one or more target cells. When the complex is formed, it affects a change in metabolism of the one or more target cells so that results in the subject upregulating the production of two or more antibodies, one or more bispecific antibodies, and combinations thereof. Examples of a target cell include, but are not limited to: an adrenal gland cell, a B cell, a bile duct cell, a chondrocyte, a cochlear cell, a corneal cell, a dendritic cell, an endocardium cell, an endometrial cell, an endothelial cell, an epithelial cell, an eosinophil, a fibroblast, a hair follicle cell, a hepatocyte, a lymph node cell, a neutrophil, a macrophage, a mucosal cell, a myocyte, a neuron, a glomeruli cell, an optic nerve cell, an osteoblast, an ovarian tissue cell, a pancreatic islet beta cell, a pericardium cell, a platelet, a red blood cell (RBC), a retinal cell, a scleral cell, a Schwann cell, a stem cell, a T cell, a testicular tissue cell, a thyroid gland cell, an uveal cell, or combinations thereof.

Some embodiments of the present disclosure relate to a therapy that can be administered to a subject with the condition. The therapy comprises a step of administering to the subject a therapeutically effective amount of an agent that will upregulate production of two or more antibodies, one or more bispecific antibodies, and combinations thereof. When the therapy is administered to a patient, the therapy will promote the in vivo production of two or more antibodies, one or more bispecific antibodies, and combinations thereof.

Some embodiments of the present disclosure relate to a method of treating a condition where the method comprises a step of administering to the subject a therapeutically effective amount of an agent that will upregulate production of two or more antibodies, one or more bispecific antibodies, and combinations thereof.

### EXAMPLE

### Example 1

BALB/c mice were purchased from Charles River, and AAV vector administrations were performed on 6-week-old mice. An AAV vector was administered by intramuscular injection in the gastrocnemius muscle, using a 29-gauge needle in a 40-μL injection volume. AAV-mAbs were administered intramuscularly at a dose of 2×10¹¹ vector genomes per mouse. Saphenous vein blood samples were collected on a weekly basis for 1 month after AAV administration. Serum levels of an antibody to Clostridium difficile Toxin A or an antibody to Clostridium difficile Toxin B were detected and confirmed by ELISA.

### Example 2

In Example 2, the agent is an AAV6.2FF gene vector that includes a gene insert for the genes responsible for producing an anti-Clostridium difficile toxin A antibody in humans.

In this example, the gene insert for anti-Clostridium difficile toxin A produces an antibody that comprises the following base sequence for a variable heavy chain (SEQ ID NO. 1):

caggtgcaactcgtcgaaagcggcggaggcgtcgtccagccaggaagatca
ttgaggctttcttgcgcagcttcaggcttctccttcagtaactatggtatg
cactgggtccggcaggcccctgggaaagggctggaatgggttgcccttatt
tggtacgacggtagcaacgaggattacacagattcagttaagggacggttt
actatttctagggacaacagtaaaaacaccctctatcttcagatgaacagc
cttcgcgctgaagacacagccgtatattattgcgctagatgggggatggtg
cgaggtgtcatcgatgtgtttgacatttggggccaaggaactgttgttaca
gtgagtagcgcc

And, the gene insert for anti-Clostridium difficile toxin A produces an antibody that comprises the following base sequence for the variable light chain (SEQ ID NO. 2):

gacattcaaatgactcaatctccttcctccgtctccgcatccgtaggagac
cgggtgactatcacttgtcgcgccagtcagggcatatccagttggctggca
tggtaccagcataagccaggaaaagccccaaaattgctgatatatgcagca
tcaagtttgcaatccggggtgccctccaggttctctggtagtggaagcggt
acagatttcacactgacaatcagcagtcttcaacccgaggacttcgccaca
tattactgccagcaggctaacagcttcccctggacattcggccaggggacc
aaggtggaaattaaaaggacagttgctgca

### Example 3

In Example 3, the agent is an AAV6.2FF gene vector that includes a gene insert for the genes responsible for producing an anti-Clostridium difficile toxin B antibody in humans.

The gene insert for anti-Clostridium difficile toxin B produces an antibody that comprises the following base sequence for the variable heavy chain (SEQ ID NO. 3):

gaagttcagttggttcaatcaggcgctgaggttaagaaatccggggagtct
ttgaaaatcagttgtaaggggagcggatacagctttacctcctattggata
ggctgggttaggcaaatgccagggaagggcctggaatggatgggaatattc
taccctggggattcatccacacggtacagtccatccttccagggtcaagtt
accatttctgctgataagagcgtgaataccgcttacttgcaatggagcagt
ctcaaagcaagcgacaccgccatgtactattgcgctcgcagaaggaattgg
ggcaatgctttcgacatatgggggcagggcactatggttactgtgtccagt
gca

And, the gene insert for anti-Clostridium difficile toxin B produces an antibody that comprises the following base sequence for the variable light chain (SEQ ID NO. 4):

gaaatagtgttgactcaatcacctggaacactgtcattgtcacccggcgaa
agggccacactgtcttgtagggccagtcagagtgtctcttcctcttacctc
gcttggtatcagcagaaacccggccaggctccccggctgcttatatatgga
gctagttcccgcgctacaggtattcctgatagatttagtggaagtggtagc
ggaacagactttaccttgactatctcacgacttgaacccgaggatttcgcc
gtatattattgtcaacaatacgggtcaagtacatggactttcggtcaaggt
acaaaagttgaaatcaaacggacagttgctgcc

The invention claimed is:

1. A recombinant virus vector (RVV) comprising at least one virus and a gene insert that induces a target cell to produce two or more antibodies.

2. The RVV of claim 1, wherein each of the two or more antibodies comprises both of SEQ ID NO. 1 and SEQ ID NO. 2.

3. The RVV of claim 1, wherein each of the two or more antibodies comprises both of SEQ ID NO. 3 and SEQ ID NO. 4.

4. The RVV of claim 1, wherein at least one of the two or more antibodies comprises both of SEQ ID NO. 1 and SEQ ID NO. 2 and at least another of the two or more antibodies comprises both of SEQ ID NO. 3 and SEQ ID NO. 4.

5. The RVV of claim 1, wherein the target cell produces at least one bi-specific antibody.

6. The RVV of claim 3, wherein the at least one bi-specific antibody comprises SEQ ID NO. 1 and SEQ ID NO. 2.

7. The RVV of claim 3, wherein the at least one bi-specific antibody comprises SEQ SEQ ID NO. 3 and SEQ ID NO. 4.

8. The RVV of claim 1, wherein the RVV is of a genus that is one of a flavivirus, an influenza, an enterovirus, a rotavirus, a rubellavirus, a rubivirus, a morbillivirus, an orthopoxvirus, a varicellovirus, a dependoparvovirus, an alphabaculovirus, a betabaculovirus, a deltabaculovirus, a gammabaculovirus, a mastadenovirus, a rubulavirus, a simplexvirus, a varicellovirus, a vesiculovirus, a lyssavirus, a cytomegalovirus, or combinations thereof.

9. A method of making an agent/target cell complex, the method comprising a step of administering a recombinant virus vector (RVV) to a target cell for forming the agent/target cell complex, wherein the agent/target cell complex causes the target cell to increase a production of two or more antibodies.

10. The method of claim 9, wherein each of the two or more antibodies comprises both of SEQ ID NO. 1 and SEQ ID NO. 2.

11. The method of claim 9, wherein each of the two or more antibodies comprises both of SEQ ID NO. 3 and SEQ ID NO. 4.

12. The method of claim 9, wherein at least one of the two or more antibodies comprises both of SEQ ID NO. 1 and SEQ ID NO. 2 and at least another of the two or more antibodies comprises both of SEQ ID NO. 3 and SEQ ID NO. 4.

13. The method of claim 9, wherein the target cell also produces at least one bi-specific antibody that comprises SEQ ID NO. 1, and SEQ ID NO. 2.

14. The method of claim 9, wherein the target cell also produces at least one bi-specific antibody that comprises SEQ ID NO. 3, and SEQ ID NO. 4.

15. The method of claim 9, wherein the target cell is one or more of an adrenal gland cell; a B cell; a bile duct cell; a chondrocyte; a cochlear cell; a corneal cell; a dendritic cell, an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a stem cell, a T cell; a testicular tissue cell; a thyroid gland cell; an uveal cell; and combinations thereof.

16. A pharmaceutical agent comprising:
a. an agent that upregulates production of two or more antibodies, at least one bi-specific antibody, or combinations thereof;
b. a pharmaceutically acceptable carrier; and/or
c. an excipient.

17. The pharmaceutical agent of claim 16, wherein the pharmaceutical agent is in a solid form or a fluid form.

18. A method of treating a condition, the method comprising a step of administering to a subject a therapeutically effective amount of an agent for upregulating the subject's production of two or more human antibodies, at least one bi-specific antibody, or combinations thereof.

19. The method according to claim 18, wherein the condition is sepsis.

20. The method according to claim 18, wherein the condition is a parasite.

21. The method according to claim 18, wherein the condition is caused by a bacteria, or a toxin excreted by a bacteria.

22. The method according to claim 18, wherein the condition is an infection caused by one of a non-hemorrhagic virus, an amoeba, a mycoplasma, a fungus, a prion and combinations thereof.

23. The method according to claim 18, wherein the step of administering occurs by an intravenous route, an intramuscular route, an intraocular route, an intraperitoneal route, an intrathecal route, an intravesical route, a topical route, an intranasal route, a transmucosal route, a pulmonary route, and combinations thereof.

24. The method according to claim 18, wherein the therapeutically effective amount is between about 10 to about 1×10¹⁶ TCID₅₀/kg of the patient's body weight.

25. The method according to claim 18, wherein the therapeutically effective amount is between about 10 to about 1×10¹⁶ total particles/kg of the agent.

26. The method according to claim 18, wherein the therapeutically effective amount is between about 10 to about 1×10¹⁶ VG/kg of the agent.


SEQUENCE LISTING (4 sequences)

SEQ ID NO: 1 - DNA, 369 bp, Artificial Sequence
  Synthetic Sequence
caggtgcaactcgtcgaaagcggcggaggcgtcgtccagccaggaagatcattgaggctttcttgcgcagcttcaggcttctccttcagtaactatggtatgcactgggtccggcaggcccctgggaaagggctggaatgggttgcccttatttggtacgacggtagcaacgaggattacacagattcagttaagggacggtttactatttctagggacaacagtaaaaacaccctctatcttcagatgaacagccttcgcgctgaagacacagccgtatattattgcgctagatgggggatggtgcgaggtgtcatcgatgtgtttgacatttggggccaaggaactgttgttacagtgagtagcgcc

SEQ ID NO: 2 - DNA, 336 bp, Artificial Sequence
  Synthetic Sequence
gacattcaaatgactcaatctccttcctccgtctccgcatccgtaggagaccgggtgactatcacttgtcgcgccagtcagggcatatccagttggctggcatggtaccagcataagccaggaaaagccccaaaattgctgatatatgcagcatcaagtttgcaatccggggtgccctccaggttctctggtagtggaagcggtacagatttcacactgacaatcagcagtcttcaacccgaggacttcgccacatattactgccagcaggctaacagcttcccctggacattcggccaggggaccaaggtggaaattaaaaggacagttgctgca

SEQ ID NO: 3 - DNA, 360 bp, Artificial Sequence
  Synthetic Sequence
gaagttcagttggttcaatcaggcgctgaggttaagaaatccggggagtctttgaaaatcagttgtaaggggagcggatacagctttacctcctattggataggctgggttaggcaaatgccagggaagggcctggaatggatgggaatattctaccctggggattcatccacacggtacagtccatccttccagggtcaagttaccatttctgctgataagagcgtgaataccgcttacttgcaatggagcagtctcaaagcaagcgacaccgccatgtactattgcgctcgcagaaggaattggggcaatgctttcgacatatgggggcagggcactatggttactgtgtccagtgca

SEQ ID NO: 4 - DNA, 339 bp, Artificial Sequence
  Synthetic Sequence
gaaatagtgttgactcaatcacctggaacactgtcattgtcacccggcgaaagggccacactgtcttgtagggccagtcagagtgtctcttcctcttacctcgcttggtatcagcagaaacccggccaggctccccggctgcttatatatggagctagttcccgcgctacaggtattcctgatagatttagtggaagtggtagcggaacagactttaccttgactatctcacgacttgaacccgaggatttcgccgtatattattgtcaacaatacgggtcaagtacatggactttcggtcaaggtacaaaagttgaaatcaaacggacagttgctgcc